(19) United States
(12) Patent Application Publication
(10) Pub. No.: US 2015/0157827 A1
(43) Pub. Date: Jun. 11, 2015

(54) Catheter Tube System and Method for Producing a Catheter Tube System

(71) Applicant: Raumedic AG, Munchberg (DE)

(72) Inventor: Björn Gläsel, Kirchenlamitz (DE)

(21) Appl. No.: 14/562,704

(22) Filed: Dec. 6, 2014

(30) Foreign Application Priority Data
Dec. 6, 2013 (DE) 10 2013 225 154.8

(51) Int. Cl. A61M 25/00; B32B 37/14

(52) U.S. Cl. CPC A61M 25/0009; A61M 25/0026; B32B 37/142; A61M 2025/004; A61M 2207/00

(57) ABSTRACT

A catheter tube system has a first tube, in particular a multi-lumen tube, and at least one second tube, for example at least two single-lumen tubes. The inner lumina of the two tubes have a fluid connection to one another that is sealed in relation to an environment by means of a cured connecting compound. The result is a catheter tube system, in which the tubes are securely and tightly connected to one another.

### CROSS-REFERENCE TO RELATED APPLICATIONS

This application claims the priority of German Patent Application Serial No. DE 10 2013 225 154.8 filed on 6 Dec. 2013, pursuant to 35 U.S.C. 119(a)-(d), the content of which is incorporated herein by reference in its entirety as if fully set forth herein.

### FIELD OF THE INVENTION

The invention relates to a catheter tube system with a first tube, with at least one second tube with at least one inner lumen, the diameter of which differs from the diameter of the at least one inner lumen of the first tube, wherein the inner lumina of the two tubes have a fluid connection to one another that is sealed in relation to an environment by means of a cured connecting compound, and to a catheter tube system with a multi-lumen tube as a first tube, with at least two single-lumen tubes as second tubes, wherein each of the single-lumen tubes has a fluid connection to a lumen of the multi-lumen tube, wherein the fluid connections between the lumina of the multi-lumen tube and the single-lumen tubes are sealed in relation to one another in each case in a fluid-tight manner by means of a cured connecting compound. Furthermore, the invention relates to a method for producing a catheter tube system of this type.

### BACKGROUND OF THE INVENTION

When producing catheters, there is often the need to manufacture catheter tube systems, in which tubes, in particular micro tubes, of various diameters, have to be connected to one another or in which multi-lumen tubes, in particular micro multi-lumen tubes, have to be connected to associated feed tubes.

### SUMMARY OF THE INVENTION

An object of the present invention is to disclose catheter tube systems of this type and a production method for them.

This object is achieved according to the invention by a catheter tube system with a first tube, with at least one second tube with at least one inner lumen, the diameter of which differs from the diameter of the at least one inner lumen of the first tube, wherein the inner lumina of the two tubes have a fluid connection to one another that is sealed in relation to an environment by means of a cured connecting compound, and by a catheter tube system with a multi-lumen tube as a first tube, with at least two single-lumen tubes as second tubes, wherein each of the single-lumen tubes has a fluid connection to a lumen of the multi-lumen tube, wherein the fluid connections between the lumina of the multi-lumen tube and the single-lumen tubes are sealed in relation to one another in each case in a fluid-tight manner by means of a cured connecting compound. In relation to the production method, the object is achieved according to the invention by a method for producing a catheter tube system according to the invention, the method having the following steps: positioning the first tube and the at least one second tube, so that an end portion of the at least one lumen of the first tube is arranged adjacent to the corresponding end portion of the lumen of the at least one second tube; introducing at least one connecting body, namely a connecting tube, into the prepositioned tubes in such a way that the connecting body, on the one hand, penetrates into the end portion of one of the lumina of the first tube and, on the other hand, into the corresponding end portion of the second tube; encapsulating the connecting body in the region of the two opposing end portions with a curable connecting compound; and curing the connecting compound to produce the cured connecting compound.

It was recognised that a fluid-tight connection between the lumina of the tubes with a different inner lumen diameter or between the multi-lumen tube and the plurality of single-lumen tubes can be produced by a curable connecting compound. A fluid connection can also be produced by this between micro tubes, in other words between tubes with an inner lumen having a diameter of less than 0.5 mm, for example of 250 μm, of 150 μm, of 100 μm or even smaller inner lumen diameters. The curable connecting compound ensures the tightness of the fluid connection here. This production of the fluid connection can advantageously be used, on the one hand, to bridge different internal diameters of tubes to be connected and, on the other hand, to connect a multi-lumen tube to a corresponding number of feed tubes.

A combination of these advantages is produced in a configuration of the catheter tube system such that the diameter of at least one of the inner lumina of the single-lumen tubes differs from the diameter of the associated inner lumen of the multi-lumen tube.

In a configuration of the ready-to-use catheter tube system in which the at least one sealed fluid connection is completely provided by means of a cured connecting compound, an additional fluid connecting body is dispensed with in the finished catheter tube system. This allows fluid connections with an optimised through-flow.

Variants of the connecting compound made of at least one of the materials comprising UV adhesive, cyanoacrylate, silicone adhesive, epoxy resin, high-strength adhesive, and a mixture of solvent and adhesive have proven to be particularly suitable. Depending on the requirements made of a viscosity, a curing period, a curing temperature and a material compatibility, one of these connecting compound variants or a mixture thereof is used.

A connecting tube between the tubes, which is a component of the sealed fluid connection, does not have to be drawn from the tube system once the connecting compound has cured, which simplifies production.

A connecting tube having an external diameter that varies along its extent may be adapted to the respective internal diameters of the associated tube lumina. The connecting tube may have an internal diameter varying along its extent.

The advantages of a production system for producing a catheter tube system according to the invention by means of the following steps: positioning the first tube and the at least one second tube, so that an end portion of the at least one lumen of the first tube is arranged adjacent to the corresponding end portion of the lumen of the at least one second tube; introducing at least one connecting body, namely a connecting tube, into the prepositioned tubes in such a way that the connecting body, on the one hand, penetrates into the end portion of one of the lumina of the first tube and, on the other hand, into the corresponding end portion of the second tube; encapsulating the connecting body in the region of the two opposing end portions with a curable connecting compound; and curing the connecting compound to produce the cured connecting compound correspond to those, which have already been described above in conjunction with the catheter tube systems according to the invention.

The connecting body to be drawn from the catheter tube system once the connecting compound has cured may in turn be a tube, in particular a connecting tube. This facilitates the drawing process, as a tube to be drawn can yield with respect to its external diameter. If a connecting tube is used as a connecting body, depending on the requirements made of the ready-to-use catheter tube system, a decision can be made as to whether the connecting body is drawn or not.

Embodiments of the invention will be described in more detail below with the aid of the drawings.

### BRIEF DESCRIPTION OF THE DRAWINGS

FIG. 1 shows perspectively, positioned in an insertion body, a two-lumen tube and two associated single-lumen tubes, connected by a connecting body, which penetrates into the end portions of the respective, associated tube lumina, before an encapsulation of the connecting body with a curable connecting compound;

FIG. 2 shows, cutout-wise, in an axial longitudinal section, a further configuration of a prepositioned arrangement of a two-lumen tube and two associated single-lumen tubes with inserted connecting bodies before the encapsulation.

FIG. 3 shows, in a view similar to FIG. 2, a further configuration of the prepositioned arrangement of the two-lumen tube and two single-lumen tubes with a connecting body configured, in each case, as a connecting tube;

FIG. 4 shows, prepositioned in a view similar to FIG. 2, a single-lumen tube and an associated further single-lumen tube with another internal diameter with an additional connecting body, which in turn penetrates into the end portions of the two tubes associated with one another, before the encapsulation of the connecting body with a curable connecting compound; and

FIG. 5 shows, in a view similar to FIG. 4, the two prepositioned single-lumen tubes with a connecting body configured as a connecting tube.

### DESCRIPTION OF THE PREFERRED EMBODIMENT

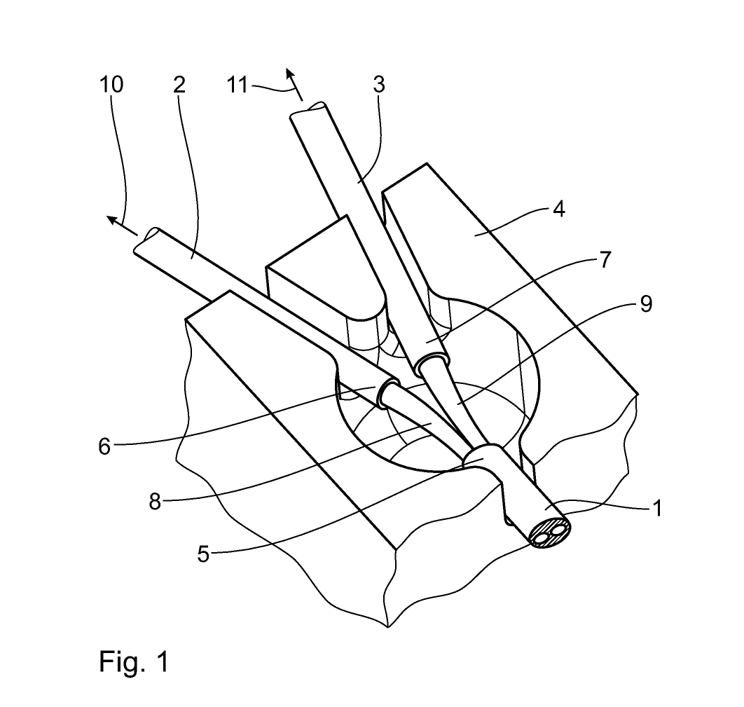

FIG. 1 shows an arrangement in a manufacturing step of a production method of a catheter tube system. A multi-lumen tube 1 in the form of a two-lumen tube, which is a first tube, and two single-lumen tubes 2, 3, in each case as second tubes, belong to the finished catheter tube system. Each of the two single-lumen tubes 2, 3 has a fluid connection to a lumen of the multi-lumen tube 1 in the finished catheter tube system.

In the manufacturing step shown in FIG. 1, the multi-lumen tube 1 and the two single-lumen tubes 2, 3 are positioned in an insertion body 4, which is simultaneously a component of a mould, in such a way that an end portion 5 of the multi-lumen tube 1 is arranged adjacent to the corresponding end portions 6, 7 of the single-lumen tubes 2, 3. The insertion body 4 is a half shell. The insertion body 4 may be an injection-moulded component.

In the manufacturing step shown in FIG. 1, two connecting bodies 8, 9 are introduced into the prepositioned tubes 1, 2 and 1, 3. The connecting body 8 is introduced into the prepositioned tubes 1, 2 in such a way that the connecting body 8, on the one hand, penetrates into the end portion 5 of the one lumen of the multi-lumen tube 1 and, on the other hand, into the corresponding end portion 6 of the one single-lumen tube 2. The second connecting body 9 is prepositioned in such a way that it penetrates, on the one hand, into the end portion 5 of the other lumen of the multi-lumen tube 1 and, on the other hand, into the corresponding end portion 7 of the other single-lumen tube 3.

After the positioning of the tubes 1 to 3 and the connecting bodies 8, 9 in the insertion body 4 according to FIG. 1, an encapsulation of the connecting bodies 8, 9 takes place in the region of the opposing end portions 5, 6 and 7 with a curable connecting compound. The curable connecting compound may be a UV adhesive, a cyanoacrylate, a silicone adhesive, an epoxy resin, a high-strength adhesive or a mixture of an adhesive and a solvent. Connecting compounds, which are assembled from a plurality of these components, are also possible.

The connecting compound is then cured.

Two variants of the connecting bodies 8, 9 are possible. The connecting bodies 8, 9 may be mandrels, which, once the connecting compound has cured, are drawn from the catheter tube system. In this case, the connecting bodies 8, 9 have, for example, end portions manufactured from wire, by means of which the connecting bodies 8, 9 can be drawn from the single-lumen tubes 2, 3 in the direction of the arrows 10, 11 shown in FIG. 1. After the mandrel connecting bodies 8, 9 have been drawn, the catheter tube system is finished, the inner lumina of the tubes 1, 2, on the one hand, and of the tubes 1, 3, on the other hand, having a fluid connection to one another that is sealed in relation to an environment by means of the cured connecting compound. The single-lumen tube 2 has a fluid connection here to one of the two lumina of the multi-lumen tube 1 and the other single-lumen tube 3 has a fluid connection to the other lumen of the multi-lumen tube 1, so the two fluid connections are sealed in relation to one another.

In the embodiment of the connecting bodies 8, 9 as a mandrel, they may be configured as solid bodies or as tube bodies.

In one variant of the production method, the connecting bodies 8, 9 are configured as connecting tubes. Once the connecting compound has cured, these connecting tubes 8, 9, between the tubes 1, 2, on the one hand, and 1, 3, on the other hand, are a component of the respective sealed fluid connection.

The connecting bodies 8, 9, along their extent, have a varying external diameter, which is adapted to the internal diameters of the associated tube lumina.

The respective inner lumina of the single-lumen tubes 2 and 3 and the inner lumina of the multi-lumen tube 1 have an internal diameter in the range between 0.1 mm and 5.0 mm. These internal diameters, depending on the requirements made of the catheter tube system, may be different. Thus, the inner lumina of the two single-lumen tubes 2, 3 may differ from one another and from the inner lumen of the multi-lumen tube 1. Alternatively, at least two of the inner lumina of the tubes 1 to 3 may also have the same internal diameter.

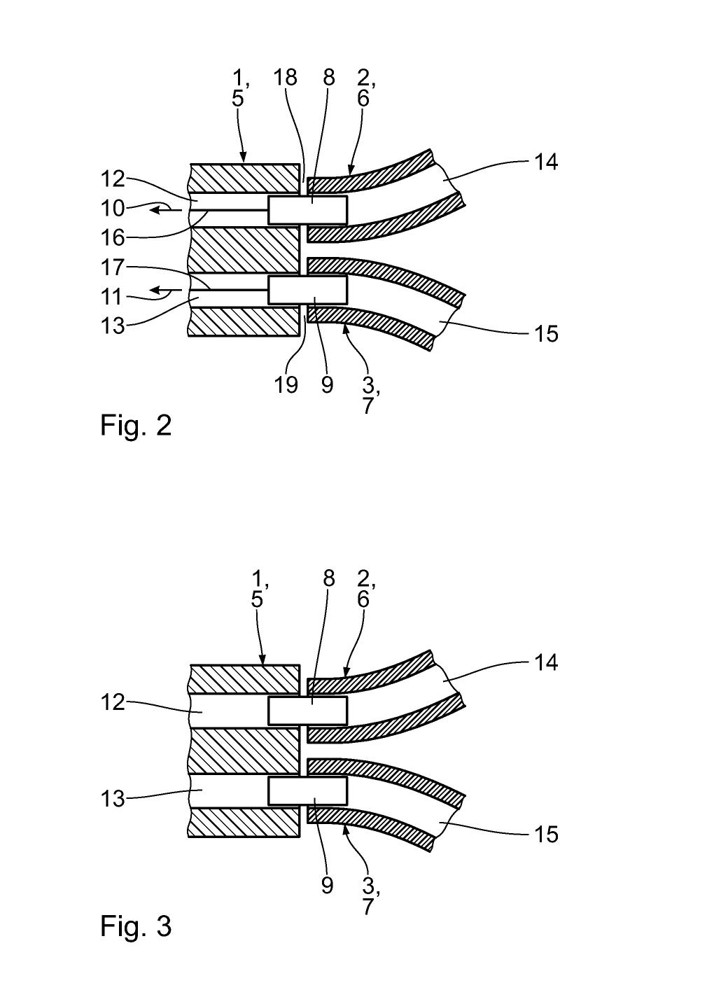

With the aid of FIG. 2, a further configuration of a catheter tube system will be described below. Components, which correspond to those, which have already been described above with reference to the configuration according to FIG. 1, have the same reference numerals and will not be discussed again in detail.

In the configuration according to FIG. 2, inner lumina 12, 13 of the multi-lumen tube 1 have the same internal diameter as the inner lumina 14, 15 of the two single-lumen tubes 2, 3. The connecting bodies 8, 9 are configured as mandrels, which can be drawn by drawing wires 16, 17 (cf. arrows 10, 11). During the encapsulation of the connecting bodies 8, 9, an intermediate space 18, 19 is closed, in each case, between the end portions 5, 6, on the one hand, and the end portions 5, 7, on the other hand, of the lumina 12, 14 or the lumina 13, 15 in a fluid-tight manner toward the environment by the cured connecting compound. The connecting compound penetrates slightly during the encapsulation into intermediate spaces between the connecting bodies 8, 9, on the one hand, and the tube end portions 5, 6 or 5, 7, on the other hand. This penetration takes place by a path, which is shorter than the extension of the connecting bodies 8, 9 into the respective end portions 5, 6 or 5, 7, so the connecting compound undesirably closing the lumina 12, 14 or 13, 15 is avoided. In addition, this penetration taking place only by a short path ensures a problem-free drawing of the connecting bodies 8, 9 by means of the drawing wires 16, 17. In order to ensure this small, on the one hand, and securely sealing, on the other hand, penetration of the connecting compound, the width of the intermediate penetration spaces, on the one hand, and, on the other hand, the viscosity of the connecting compound and a penetration period before the curing are matched to one another.

FIG. 3 shows a variant of the connecting bodies 8, 9 during the production of a variant of a catheter tube system according to FIG. 2. The connecting bodies 8, 9 are configured, in the configuration according to FIG. 3, as connecting tubes, which, once the connecting compound has cured, remain in the catheter tube system, in other words are components of the fluid connection that is outwardly sealed in each case between the inner lumina 12, 14 or between the inner lumina 13, 15. The connecting tubes 8, 9 are thus not drawn once the connecting compound has cured.

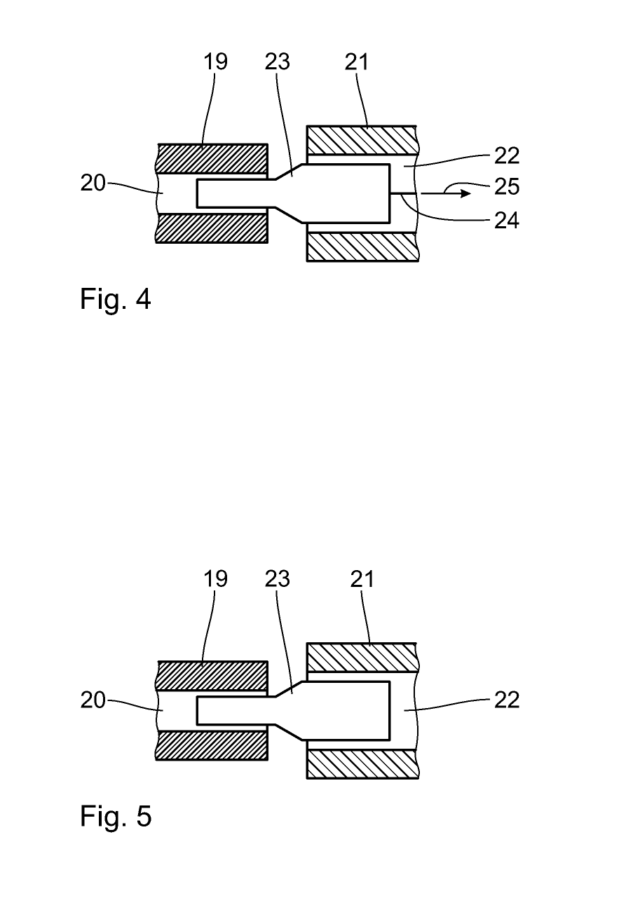

A further configuration of a catheter tube system will be described below with the aid of FIGS. 4 and 5. Components, which correspond to those, which have already been described above with reference to the configurations according to FIGS. 1 to 3, have the same reference numerals and will not be discussed again in detail.

In the catheter tube system according to FIG. 4, a first tube 19 with an inner lumen 20 has a fluid connection to a second tube 21 with an inner lumen 22. A diameter of the inner lumen 20 of the first tube 19 is smaller than a diameter of the inner lumen 22 of the second tube 21. During the production of the catheter system, end portions of the two tubes 19, 21 are prepositioned in an insertion body, not shown, in a position corresponding to that according to FIG. 4. A connecting body 23, the function of which corresponds to that of the connecting bodies 8, 9 of the configurations according to FIGS. 1 to 4, is introduced into the prepositioned tubes 19, 21, so the connecting body 23 penetrates at the end into mutually facing end portions of the two tubes 19, 21. The connecting body has an external diameter, which varies along its extent and is in each case adapted to the internal diameter of the lumina 20, 22 of the two tubes 19, 21. In the region of the end portion 19, the connecting body 23 thus has a smaller external diameter than in the region of the end portion of the second tube 21.

In the relative position of the connecting body 23 with respect to the two tubes 19, 21, which is shown in FIG. 4, the connecting body 23 is then encapsulated with the curable connecting compound and the connecting compound is cured. The connecting body, which is configured as a mandrel, is then drawn by a drawing wire 24 in the direction of the arrow 25, in accordance with that which was already stated above, in conjunction with FIGS. 1 and 2.

In the catheter system according to FIG. 5, the connecting body 23 is configured as a connecting tube, which is a component of the sealed fluid connection between the lumina 20, 22, which was produced once the connecting compound had cured. The connecting tube 23 according to FIG. 5 is thus not drawn after the encapsulation and curing of the connecting compound, but remains between the mutually facing end portions of the tubes 19 and 21.

Even if the connecting bodies 8, 9 or 23 are drawn once the connecting compound has cured, the connecting bodies 8, 9 or 23 may be configured as tubes, as this facilitates the drawing of the connecting bodies 8, 9 or 23.

1. A catheter tube system
with a first tube,
with at least one second tube with at least one inner lumen, the diameter of which differs from the diameter of the at least one inner lumen of the first tube,
wherein the inner lumina of the two tubes have a fluid connection to one another that is sealed in relation to an environment by means of a cured connecting compound.

2. A catheter tube system
with a multi-lumen tube as a first tube,
with at least two single-lumen tubes as second tubes,
wherein each of the single-lumen tubes has a fluid connection to a lumen of the multi-lumen tube,
wherein the fluid connections between the lumina of the multi-lumen tube and the single-lumen tubes are sealed in relation to one another in each case in a fluid-tight manner by means of a cured connecting compound.

3. The catheter tube system according to claim 2, wherein the diameter of at least one of the inner lumina of the single-lumen tubes differs from the diameter of the associated inner lumen of the multi-lumen tube.

4. The catheter tube system according to claim 1, wherein the at least one sealed fluid connection is completely provided by means of a cured connecting compound.

5. The catheter tube system according to claim 1, comprising a cured connecting compound of at least one of the following materials:
UV adhesive,
cyanoacrylate,
silicone adhesive,
epoxy resin,
high-strength adhesive,
a mixture of solvent and adhesive.

6. The catheter tube system according to claim 1, comprising a connecting tube between the tubes, which is a component of the sealed fluid connection.

7. The catheter tube system according to claim 6, wherein the connecting tube has an external diameter that varies along its extent.

8. A method for producing a catheter tube system according to the invention having the following steps:
positioning the first tube and the at least one second tube, so that an end portion of the at least one lumen of the first tube is arranged adjacent to the corresponding end portion of the lumen of the at least one second tube,
introducing at least one connecting body into the prepositioned tubes in such a way that the connecting body, on the one hand, penetrates into the end portion of one of the lumina of the first tube and, on the other hand, into the corresponding end portion of the second tube,
encapsulating the connecting body in the region of the two opposing end portions with a curable connecting compound,
curing the connecting compound to produce a cured connecting compound.

9. The method according to claim 8, wherein the connecting body is drawn from the catheter tube system once the connecting compound has cured.

10. The method according to claim 8 wherein the connecting body comprises a tube.

11. The method according to claim 8 wherein the lumen of the first tube has an inner diameter that is different than an inner diameter of the lumen of the at least one second tube.

12. The method according to claim 8 wherein the connecting compound is comprised of one of a UV adhesive, cyanoacrylate, silicone adhesive, epoxy resin, and a high-strength adhesive.

13. The method according to claim 8 wherein during the positioning step, introducing step, and the encapsulating step, the end portion of the lumen of the first tube and the end portion of the lumen of the at least one second tube are prepositioned adjacent one another in an insertion body.

14. The method according to claim 13 wherein the first tube comprises a multi-lumen tube having a pair of lumens and the at least one second tube comprises a pair of second tubes that each have a lumen positioned adjacent a corresponding one of the pair of the lumens of the first tube during the positioning step; in the introducing step the at least one connecting body comprising (a) a first connecting body received in the end portion of one of the lumens of the first tube and in the end portion of one of the lumens of one of the second tubes and (b) a second connecting body received in the end portion of the other one of the lumens of the first tube and in the end of the one of the lumens of the other one of the second tubes; and in the encapsulating step, encapsulating the first connecting body in a region extending between the end portion of the one of the lumens of the first tube and the end portion of the one of the lumens of the one of the second tubes and encapsulating the second connecting body in a region extending between the end portion of the other one of the lumens of the first tube and the end portion of the one of the lumens of the other one of the second tubes.

15. The method according to claim 8 wherein during the positioning step, introducing step, and the encapsulating step, the end portion of the lumen of the first tube and the end portion of the lumen of the at least one second tube are prepositioned adjacent one another in an insertion body.

16. The catheter system tube of claim 1 wherein the first tube comprises a multi-lumen tube having a pair of lumens and the at least one second tube comprises a pair of second tubes with the inner lumina of one of the lumens of the first tube connected by one fluid connection to the inner lumina of the lumen of one of the second tubes and the inner lumina of the other one of the lumens of the first tube connected by another fluid connection to the inner lumina of the lumen of the other one of the second tubes.

17. The method according to claim 16 wherein each fluid connection comprises a fluid connection provided by a cured connecting compound.

18. The method according to claim 17 wherein each fluid connection further comprises a connecting body.

19. The method according to claim 18 wherein each fluid connection comprises a tube.

20. The method according to claim 16 wherein each fluid connection comprises a connecting tube.
